(19) United States
(12) Patent Application Publication
(10) Pub. No.: US 2024/0074761 A1
(43) Pub. Date: Mar. 7, 2024

(54) IMPLANTABLE ROTATOR CUFF MUSCLE SUTURE SPACER WITH PRESSURE SENSING

(71) Applicant: National Kaohsiung University of Science and Technology, Kaohsiung City (TW)

(72) Inventors: WEN CHENG KUO, Kaohsiung City (TW); HSIANG-YU WU, Kaohsiung City (TW); SONG-CHENG HONG, Kaohsiung City (TW)

(21) Appl. No.: 18/242,781

(22) Filed: Sep. 6, 2023

(30) Foreign Application Priority Data
Sep. 6, 2022 (TW) 111133716

(51) Int. Cl. A61B 17/11; A61B 17/04

(52) U.S. Cl. CPC A61B 17/1146; A61B 17/0483; A61B 2017/00022

(57) ABSTRACT

An implantable rotator cuff muscle suture spacer with pressure sensing is provided, formed by a semiconductor manufacture procedure, including a base layer, made of a polymer material and having flexibility, and further including a first configuration region and a second configuration region, where the base layer is folded at imaginary fold line positions of the first configuration region and the second configuration region, so that the first configuration region is located above the second configuration region; a first electrode region, deposited on the first configuration region; a second electrode region, deposited on the second configuration region, corresponding to a position below the first electrode region, and configured to obtain a pressure sensing value; an inductance coil, deposited on the second configuration region and surrounding the second electrode region; and a capacitor layer, coated above a surface of the base layer to form a dielectric substance.

### CROSS-REFERENCE TO RELATED APPLICATION

This application claims the benefit of Taiwan Patent Application No. 111133716, filed on 6 Sep. 2022, which is hereby incorporated by reference for all purposes as if fully set forth herein.

### BACKGROUND

### Technical Field

The present invention relates to an implantable rotator cuff muscle suture spacer, particularly an implantable rotator cuff muscle suture spacer with pressure sensing. It can be attached to various rotator cuff muscle sutures for pressure measurement by mainly utilizing the flexible characteristic.

### Related Art

The rotator cuff muscle is of a tendon structure. It covers the shoulder joint and is specifically responsible for the rotation of the shoulder joint. However, the rotator cuff muscle tends to be ruptured due to age-related degeneration, car accident trauma, and the like, especially in the elderly aged 80 years or above. The common treatment is cuff muscle suture surgery under a shoulder arthroscopy. In the surgery, a mesh is woven with sutures to cover ruptured tendons, so the ruptured tendons are attached to the bone. However, the suture pressure in this surgery relies on the subjective judgment of a doctor. Therefore, a pressure value cannot be objectively judged. A rehabilitation therapist assisting in rehabilitation after the surgery also lacks scientific data to provide the best treatment, thereby easily leading to recurrence.

Therefore, it is necessary to provide a method for pressure sensing in such surgery. Generally, there are many principles and methods for pressure measurement. There will be many different design methods for different application fields or needs. But we know that most pressure sensors nowadays are only designed for low-pressure regions and cannot be used in high-pressure regions like the rotator cuff muscle. To be used as a medical device, such a sensor is required to pass a series of tests of, for example, biocompatibility and reliability and is specifically designed for applications in vivo. Therefore, there is a demand for a pressure sensor to be biocompatible and is compatible with high-pressure measurement.

### SUMMARY

The present invention provides an implantable rotator cuff muscle suture spacer with pressure sensing. It is used as a pressure sensor of a rotator cuff muscle suture high-pressure region, particularly for rotator cuff muscles. Of the flexible characteristic of the spacer, the pressure sensor can be implanted in uneven rotator cuff muscle suture application for pressure measurement.

The present invention relates to an implantable rotator cuff muscle suture spacer with pressure sensing, comprising: a pressure sensor, formed by a semiconductor manufacture procedure, comprising a base layer, made of a polymer material and having flexibility; an electrode layer, deposited on the base layer, where the electrode layer further comprises: a first electrode region; a second electrode region; and an inductance coil region, surrounding the second electrode region, where the first electrode region and the second electrode region are connected to each other through the inductance coil region; and a capacitor layer, coated on the base layer and above the electrode layer to form a dielectric; and an integrated spacer, attached to the pressure sensor and having a plurality of suture drill holes, where the base layer further has a fold line, and the base layer is folded to form the pressure sensor.

In an embodiment of the present invention, the first electrode region, the second electrode region, and the inductance coil mentioned above are all deposited on the base layer and are formed through a lithography and etching manufacturing procedure.

In an embodiment of the present invention, the base layer mentioned above is, but not limited to, polymer substrates such as a flexible Parylene substrate, a polyethylene glycol (PEG) substrate, a propylene glycol diacrylate (PPGDA) substrate, a polydimethylsiloxane (PDMS) substrate, a poly(methyl methacrylate) (PMMA) substrate, a poly(hydroxyethyl methacrylate) (PHEMA) substrate, and the like.

In an embodiment of the present invention, the flexible Parylene substrate mentioned above is one or a combination of Parylene C, Parylene D, and Parylene N.

In an embodiment of the present invention, the first electrode region and the second electrode region mentioned above are flexible metal film electrodes.

In an embodiment of the present invention, the base layer mentioned above is folded to form the pressure sensor having a U-shape in a plan view.

In an embodiment of the present invention, the first and second electrode regions mentioned above are disposed at corresponding upper and lower positions to form an electrode pair to obtain induced capacitance.

In an embodiment of the present invention, the capacitor layer mentioned above is changed through external pressure, so that the induced capacitance between the electrode pair is changed to obtain the pressure sensing value.

In an embodiment of the present invention, when the induced capacitance mentioned above changes, a resonant frequency is changed by utilizing an LC circuit oscillation technique to calculate the pressure sensing value.

In an embodiment of the present invention, the pressure sensor mentioned above forms mutual inductance through an external coil and the inductance coil in wireless sensing to obtain the pressure sensing value of the implantable rotator cuff muscle suture spacer with pressure sensing.

In an embodiment of the present invention, the capacitor layer is a porous polydimethylsiloxane film.

In the present invention, a flexible base is formed from a Parylene substrate, so the implantable rotator cuff muscle suture spacer with pressure sensing is implanted into human rotator cuff muscles for suture application. Then, a vacuum chamber currently used is replaced with the dielectric formed by the porous polydimethylsiloxane film, so that packaging design can be simplified and the disadvantage that a traditional capacitive pressure sensor can only perform measurement for the low-pressure regions is overcome, and pressure detection can be carried out externally by using the wireless sensing characteristic of an L-C resonant circuit.

### BRIEF DESCRIPTION OF THE DRAWINGS

FIG. 1 is a schematic diagram of an implantable rotator cuff muscle suture spacer with pressure sensing according to the present invention.

FIG. 2A is a schematic diagram of a pressure sensor of an implantable rotator cuff muscle suture spacer with pressure sensing according to the present invention.

FIG. 2B is a schematic diagram of another pressure sensor of an implantable rotator cuff muscle suture spacer with pressure sensing according to the present invention.

FIG. 3 is a schematic diagram of a pressure-sensing circuit of an implantable rotator cuff muscle suture spacer with pressure sensing according to the present invention.

FIG. 4 is a cross-sectional view of a base layer in a manufacturing method for an implantable rotator cuff muscle suture spacer with pressure sensing according to the present invention.

FIG. 5 is a cross-sectional view of an electrode layer in manufacturing an implantable rotator cuff muscle suture spacer with pressure sensing according to the present invention.

FIG. 6 is a cross-sectional view of a capacitor layer in manufacturing an implantable rotator cuff muscle suture spacer with pressure sensing according to the present invention.

FIG. 7 is a cross-sectional view of a pressure sensor in a folded state of an implantable rotator cuff muscle suture spacer with pressure sensing according to the present invention.

FIG. 8 is a schematic diagram of the implantation of a pressure sensor of an implantable rotator cuff muscle suture spacer with pressure sensing according to the present invention.

### DETAILED DESCRIPTION

Embodiments of the present invention are described in detail below with reference to the accompanying drawings. The accompanying drawings are mainly simplified schematic diagrams, only schematically illustrating the present invention's basic structure. Therefore, only the elements related to the present invention are labeled in these drawings. The elements shown are not drawn by the number, shape, dimensional proportion, or the like at the implementation time. The actual specifications and dimensions at the time of implementation are a selective design, and the layout pattern of elements may be more complicated.

The following embodiments are described with reference to the accompanying drawings to illustrate specific embodiments upon which the present invention can be implemented. The directional terms mentioned in the present invention, such as “up”, “down”, “front”, “back”, “left”, “right”, “inside”, “outside”, “side”, and the like are only directions with reference to the accompanying drawings. Therefore, the directional terms used are intended to illustrate and explain the present application, rather than limiting the present application. In addition, in this description, unless explicitly described to the contrary, the word “include/including” is to be understood as meaning including the element, but does not exclude any other elements.

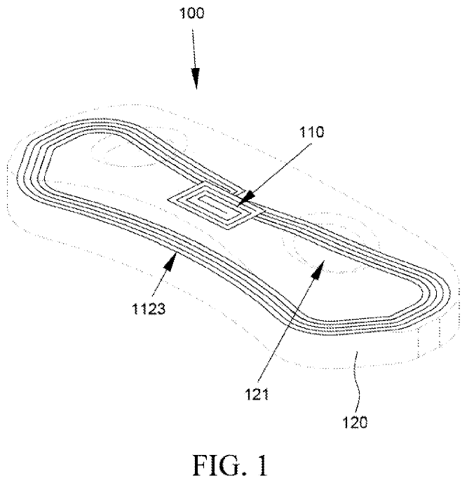

FIG. 1 is a schematic diagram of an implantable rotator cuff muscle suture spacer with pressure sensing according to the present invention. In FIG. 1, provided is an implantable rotator cuff muscle suture spacer 100 with pressure sensing, including a pressure sensor 110, formed by a semiconductor manufacture procedure, including a base layer (not shown), made of a polymer material and having flexibility; an electrode layer (not shown), deposited on the base layer, where the electrode layer further includes: a first electrode region (not shown); a second electrode region (not shown); and an inductance coil region 1123, surrounding the second electrode region, where the first electrode region and the second electrode region are connected to each other through the inductance coil region; and a capacitor layer (not shown), coated on the base layer and above the electrode layer to form a dielectric; and an integrated spacer 120, attached to the pressure sensor 110 and has a plurality of suture drill holes 121.

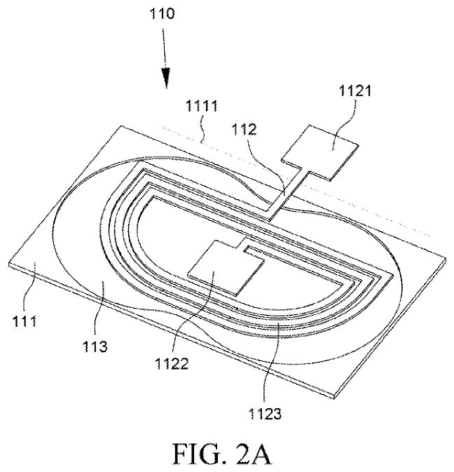

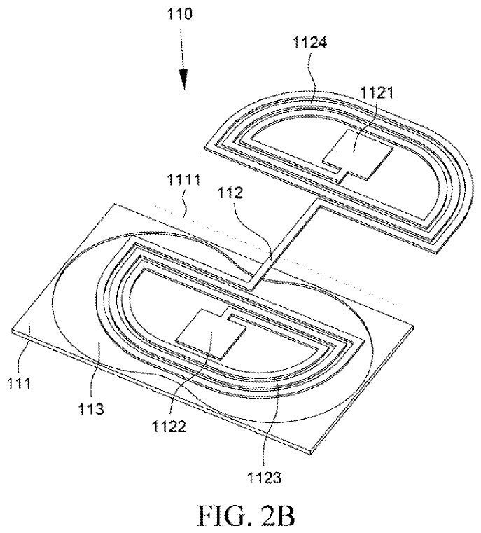

Refer to both FIG. 2A and FIG. 2B, FIG. 2A is a schematic diagram of a pressure sensor of an implantable rotator cuff muscle suture spacer with pressure sensing according to the present invention. FIG. 2B is a schematic diagram of another pressure sensor of an implantable rotator cuff muscle suture spacer with pressure sensing according to the present invention, in FIG. 2A, the pressure sensor 110 is formed by a semiconductor manufacture procedure and includes a base layer 111, made of a polymer material and having flexibility; an electrode layer 112, deposited on the base layer 111, where the electrode layer 112 further includes a first electrode region 1121; a second electrode region 1122; and an inductance coil region 1123 of a single coil design, surrounding the second electrode region 1122, where the first electrode region 1121 and the second electrode region 1122 are connected to each other through the inductance coil region 1123; and a capacitor layer 113, coated on the base layer 111 and above the electrode layer 112 to form a dielectric, where the base layer 111 further has a fold line 1111, and the base layer 111 is folded to form the pressure sensor 110.

In this embodiment, the first electrode region, the second electrode region, and the inductance coil are all deposited on the base layer and are formed through a lithography and etching manufacturing procedure.

In this embodiment, the base layer is. Still, not limited to, polymer substrates such as a flexible Parylene substrate, a polyethylene glycol (PEG) substrate, a propylene glycol diacrylate (PPGDA) substrate, a polydimethylsiloxane (PDMS) substrate, a poly(methyl methacrylate) (PMMA) substrate, a poly(hydroxyethyl methacrylate) (PHEMA) substrate, and the like.

The flexible Parylene substrate is one or a combination of Parylene C, Parylene D, and Parylene N.

In this embodiment, the first electrode region and the second electrode region are flexible metal film electrodes.

In this embodiment of the present invention, the base layer is folded to form the pressure sensor having a U-shape in a plan view.

In this embodiment, the first and second electrode regions are disposed at corresponding upper and lower positions to form an electrode pair to obtain induced capacitance.

The capacitor layer is changed through external pressure to change the induced capacitance between the electrode pair to obtain the pressure-sensing value.

When the induced capacitance changes, a resonant frequency is changed using an LC circuit oscillation technique to calculate the pressure sensing value.

In this embodiment, the pressure sensor forms mutual inductance through an external coil and the inductance coil through wireless sensing to obtain the pressure sensing value of the implantable rotator cuff muscle suture spacer with pressure sensing.

The inductance coil is a superimposed coil with a plurality of turns.

In this embodiment, the capacitor layer is a porous polydimethylsiloxane (PDMS) film.

As shown in FIG. 2B, in another embodiment, a second inductance coil region 1124 of a dual coil design is further included, surrounding the first electrode region 1121.

As for the inductance coil region 1123 of the single coil design, the number of turns of the inductance coil is 3.

As for the second inductance coil region 1124 of the dual coil design, the number of turns of the inductance coil is 3 at the top and 3 at the bottom.

Preferably, the inductance coil of the single coil design and the inductance coil of the dual coil design have an inductance line width of 200 μm.

Preferably, the inductance coil of the single coil design and the inductance coil of the dual coil design have inductance line spacing of 200 μm.

Preferably, the inductance coil of the single coil design and the inductance coil of the dual coil design have a capacitance plate width of 3 mm.

Preferably, the inductance coil of the single coil design and the inductance coil of the dual coil design have a capacitor plate length of 3 mm.

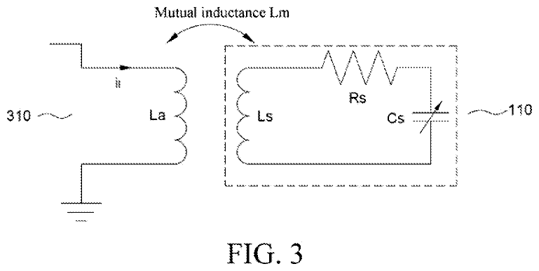

FIG. 3 is a schematic diagram of a pressure-sensing circuit of an implantable rotator cuff muscle suture spacer with pressure sensing according to the present invention. In FIG. 3, the pressure sensor forms mutual inductance through an external readout coil 310 and the pressure sensor 110. By this method, the resonant frequency transmitted by the L-C resonant circuit can be inferred, and wireless transmission signals can be captured without an additional power supply.

In this embodiment, as for the L-C resonant circuit including the external readout coil and the pressure sensor, mutual inductance is formed between the two coils when the pressure sensor is placed on the external readout coil. The inductance inside the pressure sensor is equivalent to a reflective load to the external readout coil. Therefore, the resonant frequency of the pressure sensor can be obtained through this mode. The changes in the resonant frequency caused by pressure changes can be extracted to read a signal of pressure changes.

In this embodiment, when a capacitor plate spacing changes under pressure, the change in capacitance can be expressed as ΔC, and an initial value of the capacitor plate spacing is expressed as Dₛ_{₀}. A changed distance therebetween under external stress is expressed as ΔD. In this case, the spacing will become Dₛ_{₁}. The spacing decreases, the capacitance value of the capacitor plate will increase, and the resonant frequency f_{S} will change. The change in spacing of the L-C resonant circuit subjected to pressure stress can be known through the change in the resonant frequency.

The capacitance change of a capacitor plate under pressure can be derived from the following formula:

$${D}_{{S}_{1}} = {D}_{{S}_{0}} - Δ D \\ Δ C = {C}_{{S}_{1}} - {C}_{{S}_{0}} \\ Δ C = \frac{{kE}_{0} A \cdot [ {D}_{{S}_{0}} - {D}_{{S}_{1}} ]}{{D}_{{S}_{0}} - {D}_{{S}_{1}}}$$

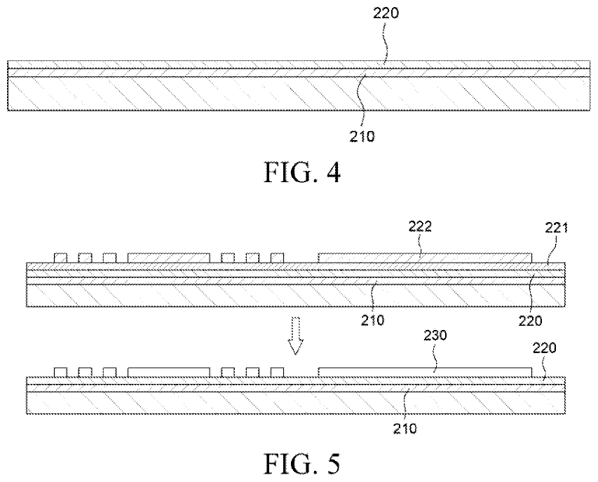

The present invention includes a manufacturing method for an implantable rotator cuff muscle suture spacer with pressure sensing. Firstly, refer to FIG. 4. FIG. 4 is a cross-sectional view of a base layer in a manufacturing method for an implantable rotator cuff muscle suture spacer with pressure sensing according to the present invention. A base layer 220 is deposited on a photoresist layer 210 first.

In this embodiment, the base layer is. Still, not limited to, polymer substrates such as a flexible Parylene substrate, a polyethylene glycol (PEG) substrate, a propylene glycol diacrylate (PPGDA) substrate, a polydimethylsiloxane (PDMS) substrate, a poly(methyl methacrylate) (PMMA) substrate, a poly(hydroxyethyl methacrylate) (PHEMA) substrate, and the like.

The flexible Parylene substrate is one or a combination of Parylene C, Parylene D, and Parylene N.

Next, refer to FIG. 5. FIG. 5 is a cross-sectional view of an electrode layer in manufacturing an implantable rotator cuff muscle suture spacer with pressure sensing according to the present invention. A chromium/gold layer 221 is first sputtered onto the base layer 220. Then, a patterned AZ-P4620 photoresist 222 is coated onto the chromium/gold layer 221. A first electrode region, a second electrode region, and an electrode layer 230 of an inductance coil are formed through a lithography and etching manufacture procedure and are deposited onto the base layer 220.

In this embodiment, an adhesion between gold and the base layer is increased by sputtering chromium.

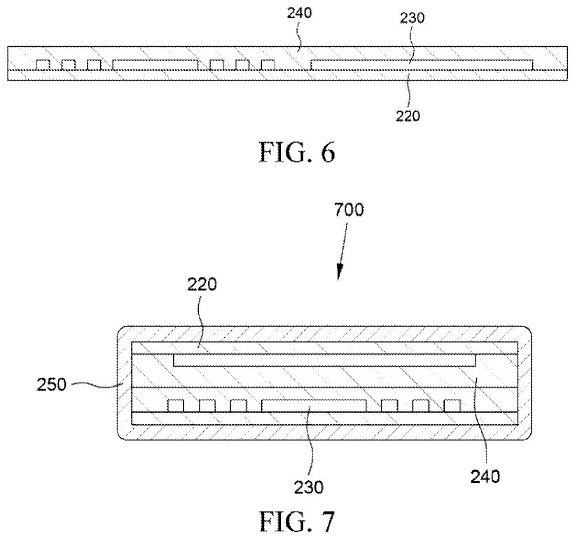

Next, refer to FIG. 6. FIG. 6 is a cross-sectional view of a capacitor layer in manufacturing an implantable rotator cuff muscle suture spacer with pressure sensing according to the present invention. A rotary coating machine is used to make a premixture of polydimethylsiloxane and a foaming agent into a capacitor layer 240, which is evenly coated onto the finished base layer 220 and electrode layer 230, and the photoresist layer is removed.

In this embodiment, ammonium bicarbonate (NH4HCO3) powder is mixed in a polydimethylsiloxane prepolymer and a curing agent first, and then the prepolymer is roasted at 90° C. for 1 h.

Since the ammonium bicarbonate is decomposed into ammonia, water and carbon dioxide simultaneously, voids are formed in a polydimethylsiloxane film, resulting in a porous polydimethylsiloxane film.

Finally, refer to FIG. 7. FIG. 7 is a cross-sectional view of a pressure sensor in a folded state of an implantable rotator cuff muscle suture spacer with pressure sensing according to the present invention. The pressure sensor 200 folded includes the base layer 220, the electrode layer 230, and the capacitor layer 240, and the pressure sensor 200 is packaged by utilizing a Parylene layer 250.

The pressure sensor 200 packaged is attached to the integrated spacer to form the implantable rotator cuff muscle suture spacer with pressure sensing.

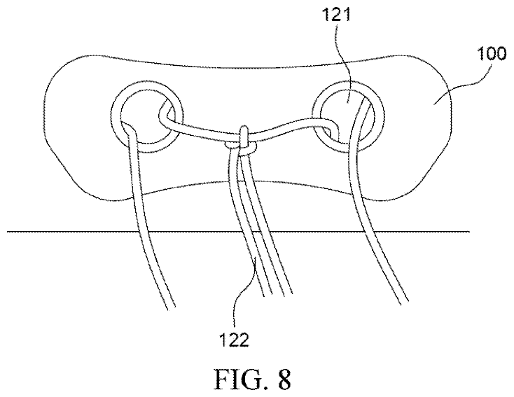

FIG. 8 is a schematic diagram of implantation of a pressure sensor of an implantable rotator cuff muscle suture spacer with pressure sensing according to the present invention. As shown in FIG. 8, the implantable rotator cuff muscle suture spacer 100 is sewn onto the rotator cuff muscle through the suture drill holes 121 with sutures 122 for suture application.

In summary, according to the implantable rotator cuff muscle suture spacer with pressure sensing, a flexible base is formed from the Parylene substrate, so that the implantable rotator cuff muscle suture spacer with pressure sensing can be implanted into human rotator cuff muscles for suture application. Then, a vacuum chamber currently used is replaced with the dielectric formed by the porous polydimethylsiloxane film, so that the packaging design can be simplified and the disadvantage that a traditional capacitive pressure sensor can only perform measurement for the low-pressure regions is overcome, and pressure detection can be carried out externally by using the wireless sensing characteristic of the L-C resonant circuit.

Although the present invention is described as above with the foregoing embodiment. The changes and embellishments made by those skilled in the art, without departing from the spirit and scope of the present invention, are still within the protection scope of the present invention.

What is claimed is:

1. An implantable rotator cuff muscle suture spacer with pressure sensing, comprising:
a pressure sensor, formed by a semiconductor manufacture procedure, comprising:
a base layer, made of a polymer material, and having flexibility;
an electrode layer, deposited on the base layer, wherein the electrode layer further comprises:
a first electrode region;
a second electrode region; and
an inductance coil region, surrounding the second electrode region, wherein the first electrode region and the second electrode region are connected to each other through the inductance coil region; and
a capacitor layer, coated on the base layer and above the electrode layer to form a dielectric; and
an integrated spacer, attached to the pressure sensor, and having a plurality of suture drill holes,
wherein the base layer further has a fold line, and the base layer is folded to form the pressure sensor.

2. The implantable rotator cuff muscle suture spacer with pressure sensing according to claim 1, wherein the base layer is a flexible Parylene substrate.

3. The implantable rotator cuff muscle suture spacer with pressure sensing according to claim 2, wherein the flexible Parylene substrate is one or a combination of Parylene C, Parylene D, and Parylene N.

4. The implantable rotator cuff muscle suture spacer with pressure sensing according to claim 1, wherein the first electrode region and the second electrode region are a flexible metal film electrode.

5. The implantable rotator cuff muscle suture spacer with pressure sensing according to claim 1, wherein the base layer is folded to form the pressure sensor having a U-shape in a plan view.

6. The implantable rotator cuff muscle suture spacer with pressure sensing according to claim 5, wherein the first electrode region and the second electrode region mentioned above are disposed at corresponding upper and lower positions to form an electrode pair to obtain induced capacitance.

7. The implantable rotator cuff muscle suture spacer with pressure sensing according to claim 6, the capacitor layer is changed through external pressure, so that the induced capacitance between the electrode pair is changed to obtain a pressure sensing value.

8. The implantable rotator cuff muscle suture spacer with pressure sensing according to claim 7, wherein when the induced capacitance changes, a resonant frequency is changed by utilizing an LC circuit oscillation technique to calculate the pressure sensing value.

9. The implantable rotator cuff muscle suture spacer with pressure sensing according to claim 1, wherein the pressure sensor forms mutual inductance through an external coil and the inductance coil in a manner of wireless sensing to obtain the pressure sensing value of the implantable rotator cuff muscle suture spacer with pressure sensing.

10. The implantable rotator cuff muscle suture spacer with pressure sensing according to claim 1, wherein the capacitor layer is a porous polydimethylsiloxane film.
